(19) United States
(12) Patent Application Publication
(10) Pub. No.: US 2013/0189765 A1
(43) Pub. Date: Jul. 25, 2013

(54) ALGAL BIO-FLOCCULATION BY INACTIVATION OF PHOTORECEPTORS

(71) Applicants: Angela Falciatore, Paris (FR); Raffaella Raniello, Napoli (NA) (IT); Chris Bowler, Paris (FR)

(72) Inventors: Angela Falciatore, Paris (FR); Raffaella Raniello, Napoli (NA) (IT); Chris Bowler, Paris (FR)

(73) Assignee: STAZIONE ZOOLOGICA "ANTON DOHRN", Napoli (NA) (IT)

(21) Appl. No.: 13/876,125

(22) PCT Filed: Oct. 14, 2011
(86) PCT No.: PCT/EP2011/068018

§ 371 Date: Mar. 26, 2013

(30) Foreign Application Priority Data
Oct. 15, 2010 (EP) 10187730.6

(51) Int. Cl. C12N 1/02

(52) U.S. Cl. CPC C12N 1/02
USPC 435/257.1; 426/61; 210/620

(57) ABSTRACT

The present invention relates to a method to induce alga and/or diatom cell bio-flocculation comprising inactivation of the expression of at least one photoreceptor in said alga and/or diatom, constructs able to inhibit the expression of at least one photoreceptor and uses thereof.

### FIELD OF THE INVENTION

The present invention relates to a method to induce alga and/or diatom cell spontaneous flocculation comprising inactivating the expression of a photoreceptor in said alga and/or diatom, to constructs able to inhibit the expression of the photoreceptor and uses thereof.

### BACKGROUND ART

Diatoms are unicellular photosynthetic eukaryotes found in most humid environments and open water masses that play a major role in the global cycling of carbon and silicon (Falkowski, 1998). They are thought to have arisen from a secondary endosymbiotic event between three eukaryotes, a red alga, a green alga, and a flagellated heterotroph. Recent analysis of diatom genomes indeed reveals a ‘mosaic’ nature, with genes derived from plant, animal and bacterial lineages (Armbrust, et al., 2004, Bowler, et al., 2008). Diatom cells therefore have a range of features that make them highly divergent from the classical cellular structures of higher plants and animals, so studies of diatom biology promise to reveal many novel aspects of commercial interest. Basic studies of diatom cell biology have nonetheless been hampered in the past by the lack of a model species and associated molecular tools. This situation is now changing following the emergence of two species, Thalassiosira pseudonana and Phaeodactylum tricornutum, that are finally beginning to yield the molecular secrets of diatoms (Armbrust, et al., 2004, Bowler, et al., 2008). Recent advances in molecular genomics, such as the development of generic Gateway-based transformation vectors for overexpression (Siaut et al., 2007) and RNAi-based gene knockdown approaches (De Riso et al., 2009) can also facilitate the use of diatom specific genes or pathways for biotechnology. Techniques to grow P. tricornutum heterotrophically have also been developed and pave the way for large-scale cultivation using microbial fermentation technology (Zaslayskaia, et al., 2001).

Biologically active compounds extracted from diatom cells have been proposed for a range of biotechnological applications (for a review see Lebeau and Robert 2003). Some diatoms synthesize the nutritionally important eicosapentaenoic (EPA) and docosahexaenoic (DHA) acids as well. PUFAs (e.g., polyunstaurated fatty acids, PUFAs) generally constitute around 25% of the total synthesized fatty acids in diatoms (Dunstan et al. 1994), and the total content can be as high as 50% of total biomass (Chisti 2007; 2008). Because some diatoms can now be genetically modified, it is possible that strains capable of synthesizing higher levels of specific PUFAs can be generated. The production of commercially desirable PUFAs could be significantly enhanced using such methods. Furthermore, diatoms are nutritionally suitable as feedstock for the mariculture industry, and they may be useful for the bioremediation of water contaminated with heavy metals or enriched in phosphate and nitrogen.

Because of their high lipid content and fatty acid composition, diatoms are also a promising source of biofuels. In fact oil productivity in many diatoms greatly exceeds the oil productivity of the best oil producing crops, making them interesting for commercial exploitation (Carrieri et al. 2008; Dismukes et al. 2008). Moreover the biotechnological exploitation of diatoms has several advantages over plants. Diatoms are able to grow at high rates thanks to their nutrient uptake system and can grow on waste, brackish, and sea water. Furthermore, biomass yields are five to ten times higher than plants because the turnover times of diatom cells are much faster, and energy yields can be between six and twelve times higher (up to 60 T/ha). Additionally, because they do not produce lignin or cellulose, as plants do, the non-lipid fraction can be used as a high protein feed in the livestock and poultry industries.

Several factors have impeded up to now the exploitation of diatoms for large scale commercial applications (Chisti 2007; 2008). In addition to the lack of efforts devoted to developing the appropriate engineering technologies, the strains that could be used are essentially wild type strains that have not been optimized for lipid production. Natural populations therefore need to be screened for high oil production and appropriate strains need to undergo mutagenesis or genetic engineering programs to improve their suitability for commercial scale production.

Genetic and metabolic engineering are likely to have the greatest impact on improving the economics of diatom biofuel production (Chisti 2007, 2008). Molecular level engineering can be used to:

1. increase photosynthetic efficiency to enable increased biomass;
2. enhance biomass growth rate;
3. increase oil content of biomass;
4. improve temperature tolerance to reduce the expense of cooling;
5. extend photosynthetic yields and reduce photoinhibition at higher light intensities;
6. identify factors that stimulate oil production;

In addition, all the production systems are needed to optimize harvesting of cells for the subsequent recovery of products/molecules of interest.

The basic concept of extensive microalgal exploitation in biotechnology is the use of relevant biomass that would need to be concentrated and recovered from the growth medium to allow for the ensuing industrial treatments. The biomass must be generally concentrated by an initial factor of at least about thirty-fold, requiring, for convenient industrial application, very low-cost harvesting processes such as “bioflocculation”, or “flocculation”, namely a spontaneous flocculation-sedimentation process of the algal cells, using no, or at most very little, flocculation chemicals. In fact, for the selection of the algal strains to cultivate the harvestability is one of the primary criteria. Up to now this step is very critical in the case of diatom macrocultures and the development of low-cost harvesting processes is a very relevant industrial goal to be realized. The controlled induction of the diatom cell autoaggregation-sedimentation, proposed in the present document is based on the inhibition of photoreceptor activity, which causes flocculation. A specific example is provided using phytochrome inactivated using an antisense strategy, alternatively inverted repeat fragment based inactivation may be used, representing a very low-cost harvesting process of interest to several industrial fields.

### SUMMARY OF THE INVENTION

In the present invention it was found that it is possible to silence the PHY gene by a construct comprising either antisense or inverted repeat fragments of the PHY gene. The transcription of such silencing constructs can be driven by various promoters, such as for example FcpBp or H4p. The introduction of the silencing constructs in P. tricornutum leads to a decrease in protein expression, preferably between 50 and 90%.

Surprisingly such cells do flocculate spontaneously very efficiently. In the present invention terms as flocculation, sedimentation, bioflocculation or aggregation are used and mean a process method whereby cells come out of suspension by means of a specific altered genotype/phenotype, i.e. because phytochrome inactivation causes the cells to aggregate and sediment. Using this method, cultured cells can be harvested easily, without addition of chemicals.

It is therefore an object of the invention a method to induce alga and/or diatom cell bio-flocculation comprising the step of inactivating the expression of at least one photoreceptor in said alga and/or diatom. Preferably the alga is selected from any of the following groups: the species Chromalveolata and Archaeplastida or the group of Bacillariophyceae; alternatively the diatom is selected from the species Thalassiosira pseudonana or Phaeodactylum tricornutum.

In a preferred embodiment the photoreceptor belongs to the group of red light photoreceptors, preferably the photoreceptor is a phytochrome, more preferably phytochrome is the Phy protein, essentially consisting of SEQ ID. No. 2, or variants thereof.

In a preferred embodiment the inactivation of the expression of the photoreceptor is achieved by means of genetic engineering, preferably comprising the step of transforming the alga and/or the diatom cell with a nucleic acid construct able to inactivate the expression of the photoreceptor. More preferably the nucleic acid construct able to inactivate the expression of the photoreceptor comprises an alga and/or diatom cell active promoter operatively linked to a nucleic acid fragment of the photoreceptor cDNA. Most preferably the nucleic acid construct is able to inactivate the expression of the photoreceptor Phy protein, essentially consisting of SEQ ID. No. 2, or variants thereof, comprising a diatom active promoter region and operatively linked to a nucleic acid fragment of the PHY cDNA comprised between nt 727 and nt 980 of SEQ ID No. 1 or between nt 727 and nt 1147 of SEQ ID No. 1 introduced in the antisense orientation with respect to the nucleic acid fragment of the PHY cDNA. In a further preferred aspect the promoter region is selected from the promoter region of the Fucoxanthin Chlorophyll a/c-binding Protein B gene (ID number 56299, SEQ ID. No. 15) or of the Histone 4 gene (ID number 34971, SEQ ID. No. 16).

It is another object of the invention a bio-flocculated alga and/or diatom cell obtainable according to the method of the invention. Preferably, the bio-flocculated alga cell is selected from the species Chromalveolata and Archaeplastida or from the group of Bacillariophyceae and the bio-flocculated diatom cell is selected from the species Thalassiosira pseudonana or Phaeodactylum tricornutum.

Such bio-flocculated alga and/or diatom cells are advantageously used for the production of biodiesel, and/or natural or recombinant molecules, and/or for the bioremediation of contaminated water or water enriched in phosphate and/or nitrogen, and/or as feed for livestock or poultry.

### DETAILED DESCRIPTION OF THE INVENTION

### Figure Legends

The present invention will be now illustrated by means of the following non limiting figures.

FIG. 1 Comparison of Phy domain organization among plants, cyanobacteria, fungi, bacteria and diatoms.

FIG. 2 Phylogenetic analysis revealing an independent Glade for diatom phytochromes (Phy) and brown algal viruses (Montsant et al., 2007).

FIG. 3 PHY cDNA sequence (SEQ ID. No. 1). The region targeted for the gene silencing is reported in grey and the primers used for the amplification of short and long antisense fragments are in bold character and underlined.

FIG. 4 Alignment of P. tricornutum (PtPHY protein) (SEQ ID No. 2) and T pseudonana (TpPHY protein) (SEQ ID No. 17) proteins.

FIG. 5 Schematic representation of antisense and inverted repeat constructs for PHY silencing.

FIG. 6 Analysis of Phy protein by immunoblot in independent silenced clones. Phy levels were quantified using a serial dilution of proteins from wild-type cells as standard and the anti-CPF1 antibody was used as loading control (A). Wild types and F-ir20, H-an4 and H-an6 mutants cultures grown under normal condition. Cell sedimentation is evident on the bottom of the flasks in the mutants (B). Optical and electron microscope analysis of silenced lines (C)

FIG. 7 Sedimentation features of the wild type (Pt1), F-ir20, H-an4 and H-an6, quantified spectrophotometrically, by following the optical density (OD) decrease over the time (up to 2 hours). The slope of the curve indicates the rate of sedimentation, as the O.D672 nm is proportional to the cell in suspension. All the mutants sediment faster than the wild types, with the H-an6 showing the fastest sedimentation rate.

### MATERIALS AND METHODS

### Cell Culture

The CCMP632 strain of Phaeodactylum tricornutum Bohlin was obtained from the culture collection of the Provasoli-Guillard National Center for Culture of Marine Phytoplancton (Bigelow Laboratory for Ocean Science-Maine, USA). Cultures were grown in f/2 medium (Guillard 1975), incubated at 18° C. under cool white fluorescent lights at ca. 100 μmol·m⁻²·s⁻¹ in a 12 h:12 h dark-light cycle.

### Antisense and Inverted-Repeat Constructs

The vectors for antisense and inverted repeat constructs were generated using standard molecular cloning procedures (Sambrook et al. 1989).

For generation of the PHY silencing vectors, the GUS antisense and inverted-repeat vectors described by De Riso et al. (2009) were used.

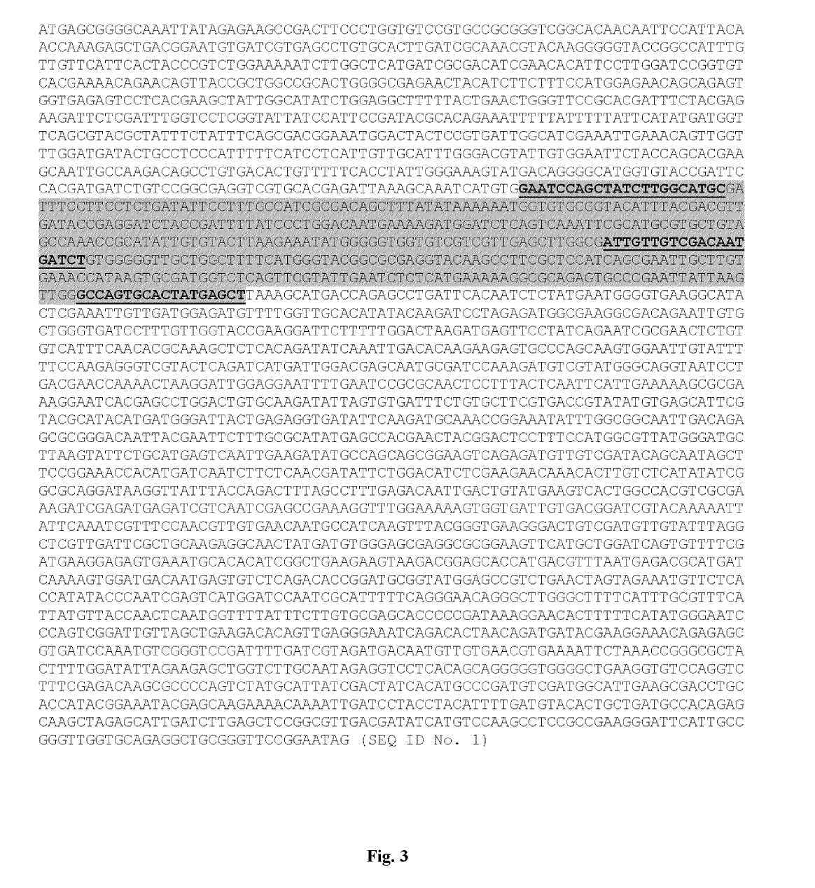

More in detail a 254 bp fragment, corresponding to the PHY gene sequence from 727 bp to 980 bp (FIG. 3), was obtained from PHY cDNA by PCR amplification with the primers GAF1FW and GAF1RV (Tab. 2), while a 421 bp product, corresponding to the region 727 bp-1147 bp (FIG. 3), was generated using GAF1FW and GAF2RV (Tab.2).

TAB. 2
Primer sequences

| | |
|---|---|
| GAF1fw | 5′-ACTGAATCCAGCTATCTTGGCATGC-3′ SEQ ID. No. 3 |
| GAF1rv | 5′-ACTTCTAGATCATTGTCGACAACAAT-3′ SEQ ID. No. 4 |
| GAF2rv | 5′-GATTCTAGAGCTCATAGTGCACTGGC-3′ SEQ ID. No. 5 |
| FcpBpfw-SacII | 5′-AGTCCGCGGAATCTCGCCTATTCATG-3′ SEQ ID. No. 6 |
| phy1fw | 5′-GCGTACGCTGAACCATCATA-3′ SEQ ID. No. 7 |
| phy1rv | 5′-GTTCCGCACGATTTCTACGA-3′ SEQ ID. No. 8 |
| phy2fw | 5′-GAATTGTGCTGGGTGATCCT-3′ SEQ ID. No. 9 |
| phy2rv | 5′-GTGAGAGCTTTGCGTGTTGA-3′ SEQ ID. No. 10 |
| phy3fw | 5′-GACATCGGGCATGTGATAGT-3′ SEQ ID. No. 11 |
| phy3rv | 5′-GCAATAGAGGTCCTCACAGCA-3′ SEQ ID. No. 12 |
| RPSfw | 5′-GTGCAAGAGACCGGACATACC-3′ SEQ ID. No. 13 |
| RPSrv | 5′-CGAAGTCAACCAGGAAACCAA-3′ SEQ ID. No. 14 |

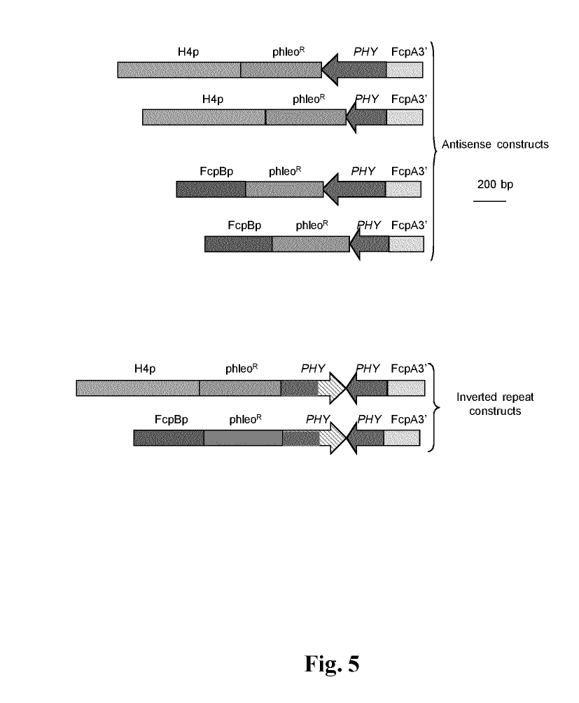

These two fragments had the first 254 bp in common. For the antisense construct, either the longer or the shorter PHY fragment was digested by EcoRI and XbaI and subsequently introduced in the antisense orientation, between the Sh ble gene and the FcpA terminator, into the EcoRI-XbaI linearized GUS antisense vector, replacing the GUS fragment (FIG. 3). For the inverted-repeat construct, both the longer and the shorter fragments were digested by EcoRI and XbaI and subsequently ligated to each other, by means of the XbaI site, in sense and antisense orientations, respectively, and were cloned into the EcoRI linearized inverted-repeat vector (FIG. 5).

Genetic Transformation of P. tricornutum

The antisense and inverted-repeat vectors were introduced into the CCMP632 strain of P. tricornutum Bohlin by microparticle bombardment, using a Biolistic PDS-1000/He Particle Delivery System (Bio-Rad, Hercules, Calif., USA) as described in (Falciatore et al. 1999).

Putative silenced clones were first selected on 50% fresh seawater (SW) agar plates (1% agar) supplemented with 50 μg/ml phleomycin (InVitrogen, San Diego, Calif., USA). After ca. three weeks, individual resistant colonies were restreaked on 50% SW agar plates supplemented with phleomycin and then inoculated into liquid f/2 medium to be further analyzed.

### Screening of Transformed Clones and Identification of Silenced Strains

In order to confirm the presence of the phy silencing constructs, an initial PCR screening of phleomicyn resistant clones was performed directly on diatom colonies (Falciatore et al. 1999). In particular a forward primer complementary to the FcpBp promoter has been used (FcpBpfw-SacII) and, as reverse primer complementary to the GAF domain GAF1fw (Tab.2). Amplification products were checked by electrophoresis on agarose gels. Afterwards a western blot analysis was performed on PCR positive clones to check the real amount of Pt Phy protein in comparison to the wild type. Proteins were extracted from a pellet corresponding to ca. 10⁷ cells in the exponential phase of growth, suspended in 45 μl of extraction buffer (Tris-HCl 50 mM pH 6.8, SDS 2%), vortexed and incubated at RT for 30 min. The soluble phase was recovered by centrifuging at 10,000×g for 30 min at 4° C. The protein extract was spectrophotometrically quantified using the BCA reagent (BCA Protein Assay kit; Thermo Scientific) in accordance to the manufacturer's instructions. 50 μg of total proteins were separated in a 10% sodium dodecyl sulphate-polyacrylamide gel electrophoresis (SDS_PAGE) and transferred to a nitrocellulose membrane (POTRAN BA85; Schleicher & Schuell BioScence GmbH, Whatman Group, Dassel, Germany). The membrane was saturated with PBS 1×, Tween 0.1%, lyophilized milk 5% (1 h at RT), then first incubated overnight with rabbit anti-Phy antibody (dilution 1:1,000) (DeRiso et al. 2009) and subsequently with goat (HRP)-conjugated anti-rabbit secondary antibody IgG (H+L) (dilution 1:10,000) (Promega), for 1 h at RT. Phy was revealed by using an Amersham enhanced chemiluminescence kit (ECL kit; Amersham Biosciences, Piscataway, N.J., USA). The anti-Cpf1 (antibody was utilized as loading control).

### Microscopic Observation of Silenced Clones

The most strongly silenced clones were inoculated into liquid f/2 medium supplemented with 50 μg/ml phleomycin and incubated at 18° C. under cool white fluorescent lights (either 100 or 25 μmol·m⁻²·s⁻¹), in a 12 h:12 h dark-light cycle.) Cell cultures were observed by optical and SEM electron microscopes (Electron Microscope Service SZN).

### Sedimentation Features

Sedimentation rate was quantified spectrophotometrically, by following the optical density (OD) decrease over the time (up to 2 hours) of the wild-type and the mutants, as the O.D₆₇₂ nm is proportional to the cell in suspension. The sedimentation rate was calculated for each strain considering that 100% of the cells were in suspension at the time 0. The OD was measured every 15 minutes for 2 hours. The slope of the curve indicates the rate of sedimentation.

### Results

### phy Knock-Down Mutants

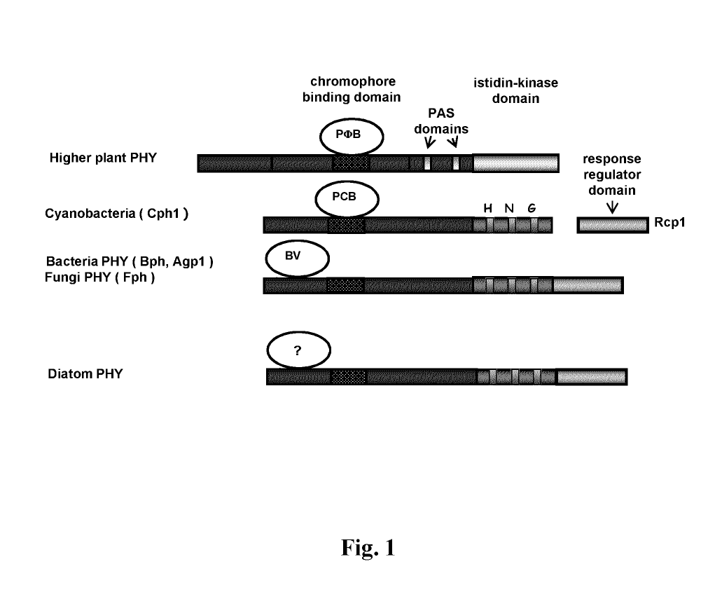

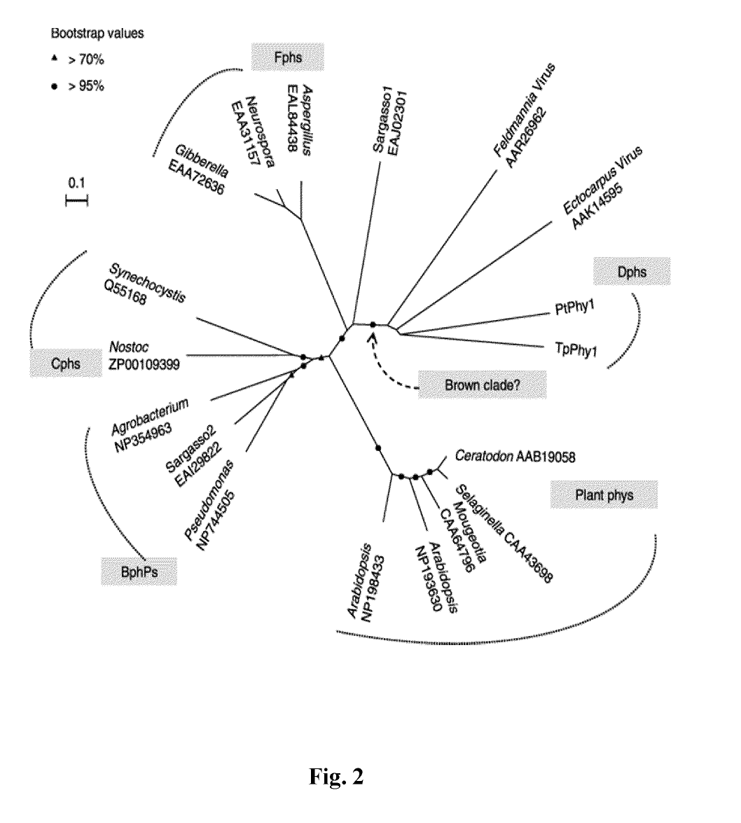

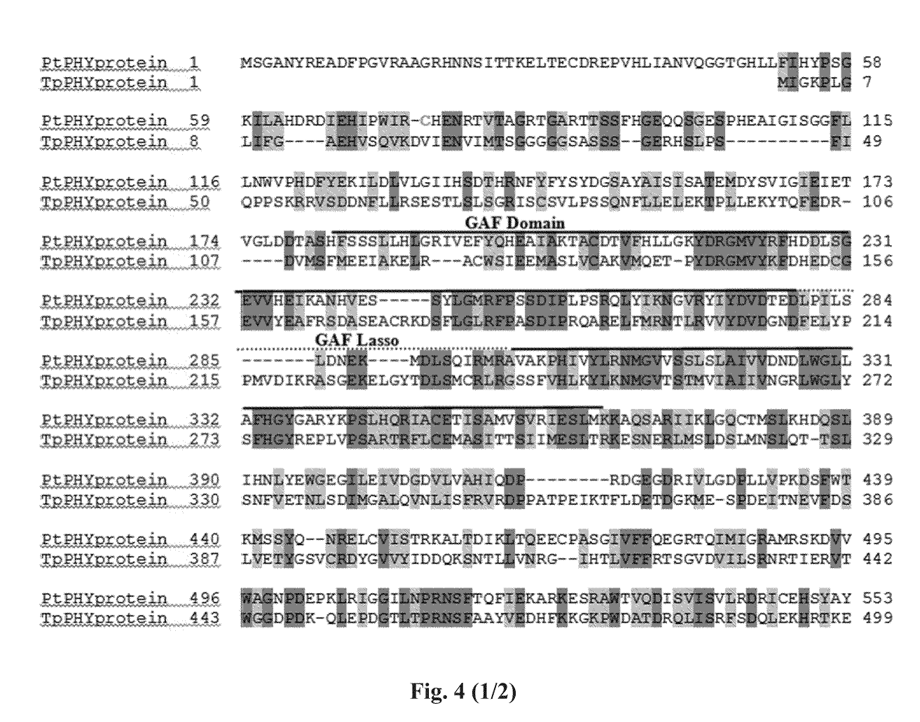

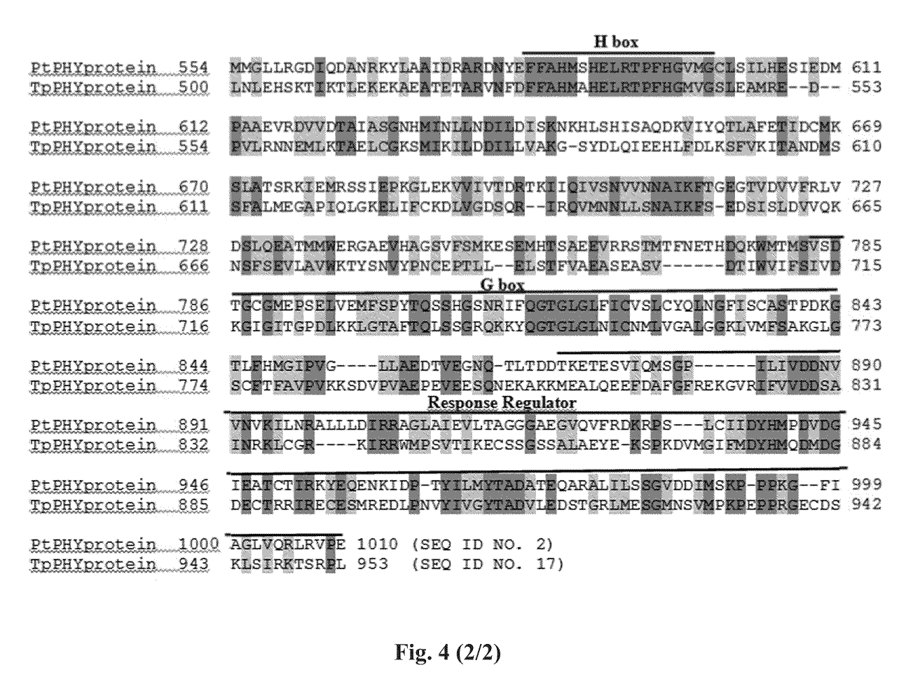

Phytochromes (Phys) belong to an extended family of photoreceptors widely distributed in photosynthetic and non-photosynthetic bacteria, fungi, algae and higher as well lower plants, living in very different environments (Montgomery and Lagarias 2002). Typically, red/far red responses are mediated by two photoconvertible phytochrome forms: Pr and Pfr, absorbing, respectively, red and far-red light (Chen et al. 2004). The overall similarity between phytochromes identified in different organisms appears in the GAF domain containing the putative chromophore binding site (FIG. 1). All Phys utilize a linear tetrapyrrole as chromophore: plants incorporate phytochromobilin (PΦB), some cyanobacteria use phycocyanobilin (PCB), and bacteriophytochromes (BphP) contain the biliverdin precursor (BV). Recently, it has been shown that bacteriophytochromes isolated from some photosynthetic bacteria do not bind a chromophore. This loss of light sensing is replaced by a redox-sensing ability coupled to a kinase activity (Vuillet et al. 2007). Phytochrome-like proteins have been identified in the genomes of the marine diatom Phaeodactylum tricornutum (http://genome.jgi-psf.org/cgi-bin/dispTranscript?db=Phatr2&id=54330&useCoords=1) and Thalassiosira pseudonana (http://genome.jgi-psf.org/Thaps3/Thaps3.home.html). Interestingly, the analysis of the diatom protein sequence indicates a putative N-terminal chromophore binding domain, followed by histidine kinase and response regulator (RR) modules at the C-terminus (Montsant et al. 2007). This structure makes the diatom Phy more similar to bacteriophytochrome (BphP) than to plant Phy; moreover diatom Phy contains a Cys residue at the N-terminus of the protein, as in bacteriophytochrome, that might be involved in the covalent linkage of the putative chromophore allowing for a light-activated kinase similar to bacterial two-component sensors (FIGS. 2, 3 and 4) (Falciatore and Bowler, 2005). In order to clarify diatom phytochrome function, the authors have recently generated phy knock-down mutants in P. tricornutum by the RNA interference approach (De Riso et al., 2009). As reported in the methods, the authors have produced different constructs containing either antisense or inverted repeat fragments of the PHY gene (FIG. 5). In particular, a fragment from the GAF region of the PHY gene has been cloned between the 3′ end of the selectable Sh ble gene, conferring resistance to phleomycin, and the terminator region of the diatom FcpA gene (Falciatore et al. 1999). This strategy allowed for a unique transcriptional product, with a consequent more efficient and simplified screening of silenced clones by their incubation on a selective medium.

Different P. tricornutum promoters were tested to successfully drive the transcription of silencing constructs: FcpBp and H4p, promoters from the Fucoxanthin Chlorophyll a/c-binding Protein B and Histone 4 genes, respectively. Their sequence is reported below.

FcpBp sequence:
(SEQ ID. No. 15)
AATCTCGCCTATTCATGGTGTATAAAAGTTCAACATCCAAAGCTAGAACT
TTTGGAAAGAGAAAGAATATCCGAATAGGGCACGGCGTGCCGTATTGTTG
GAGTGGACTAGCAGAAAGTGAGGAAGGCACAGGATGAGTTTTCTCGAGAC
ACATAGCTTCAGCGTCGTGTAGGCTAGGCAGAGGTGAGTTTTCTCGAGAC
ATACCTTCAGCGTCGTCTTCACTGTCACAGTCAACTGACAGTAATCGTTG
ATCCGGAGAGATTCAAAATTCAATCTGTTTGGACCTGGATAAGACACAAG
AGCGACATCCTGACATGAACGCCGTAAACAGCAAATCCTGGTTGAACACG
TATCCTTTTGGGGGCCTCCGCTACGACGCTCGCTCCAGCTGGGGCTTCCT
TACTATACACAGCGCGCATATTTCACGGTTGCCAGAA
H4p sequence:
(SEQ ID. No. 16)
GCAATCTCACGCACCAGGCGCTGGAAGGGCAACTTGCGGATGAGAAGGTC
CGTGGACTTCTGGTAACGACGGATCTCACGCAGAGCGACGGTTCCAGGGC
GATAACGGTGGGGCTTCTTGACTCCTCCGGTAGCCGGAGCGGACTTGCGG
GCAGCCTTGGTGGCAAGCTGCTTGCGCGGCGCTTTGCCTCCGGTGGATTT
ACGGGCGGTTTGCTTGGTTCGGGCCATTTTGACGGTTTTTTTTACAAGAG
AAGAGTTCTTGAAATTTGTGAGGTTAAAGTGTGTGGCTTCCGCCGTAGTC
AAGGAGCGTGCGGTTGCCGATCGCACCGGTACGTTCTGTAGAAATGAACA
CAGTGTGTTGAATTGAAAGTATGGCGCAGGTATGGTGTGTGATAAGTAGC
AGCCGCGCCGAGACAAACAAACTTTGGTTTCTACGACAATCTCTGTAGAC
AAGTACTAGAAACCCGTTTGAACGAGCATAAATCTGCACCGGCAGGCCAC
CAGACATCGTTTCAACGTAATATTCTACGTAACCATTTTATCCCAGGAAA
CCTACGGCCTGTGAACCACCGAGACGGAGCACTCACAATTCGCTCTCGGC
AACAACCGACAATCGTCTTACTCACAGTCAATACCGAAAACAAACAACAG

The light-regulated FcpB promoter is routinely used to obtain strong expression of transgenes in diatoms, whereas H4p has been recently identified as a good candidate to drive constitutive expression (Siaut et al. 2007).

Concerning the inverted repeat construct, it was expected to encode for RNAs that fold into hairpin structures. In fact two PHY fragments (the 254 bp and the 421 bp fragment as indicated above) were introduced with an opposite orientation into the vector and were able to generate a loop of 167 bp after the annealing of their complementary regions of 254 bp in length (FIG. 5).

The different constructs were introduced into the CCMP632 strain of P. tricornutum by stable nuclear transformation (Falciatore et al. 1999). Putative silenced clones were initially selected on phleomycin. A higher number of clones were obtained by the transformation with constructs under FcpBp (77 clones), while only 30 clones were obtained by transforming with constructs driven by H4p (Tab. 1).

TABLE 1
Clones

| | Phleo^{R} | Western | Stable | |
|---|---|---|---|---|
| | clones | blot+ | clones | Phenotype+ |
| F-ir | 67 | 16 | 3 | 1 |
| F-an | 10 | 2 | — | — |
| H-ir | 12 | — | — | — |
| H-an | 18 | 3 | 2 | 2 |

Phleo^{R} clones: number of pheomycin resistant clones;
Western blot+: clones with a significant decrease in Phy content (revealed by wester-blot);
Stable clones: clones with an unchanged level of PHY silencing after nine months;
Phenotype+: clones with a peculiar phenotype revealed by microscope observations.
F-IR, vector containing the sense (254 bp)/antisense (421) fragments under the FcpBp;
F-AN, vector containing the antisense (421 bp) fragment under the FcpBp;
H-IR, vector containing the sense (254 bp)/antisense (421) fragments under the H4p;
H-AN, vector containing the antisense (254 bp) fragment under the H4p.

A preliminary PCR screening has been performed (as reported in M&M) to check for the presence of the silencing constructs. Afterwards, the content of Phy protein relative to that of the wild type was verified by western blot using a specific antibody in the clones that were positive in the PCR analysis.

Three clones whose transgene expression was driven by the H4 promoter (containing constructs with the antisense fragment of 254 bp) showed a significant reduction in Dph1 content by western blot. However, only in two clones, H-an6 and H-an4 (containing the 254 bp antisense) the protein reduction was stable after one year and independent of the light conditions (Tab. 1).

In the case of the 421 bp antisense constructs driven by FcpBp, two silenced clones were identified but the protein reduction was transient for both of them (Tab. 1). The highest number of positive clones were identified with the inverted repeat construct under the FcpB promoter (16 clones), but in this case three clones (F-ir15, F-ir20 and F-ir26) showed stable silencing, revealing unchanged protein levels after one year (Tab. 1),

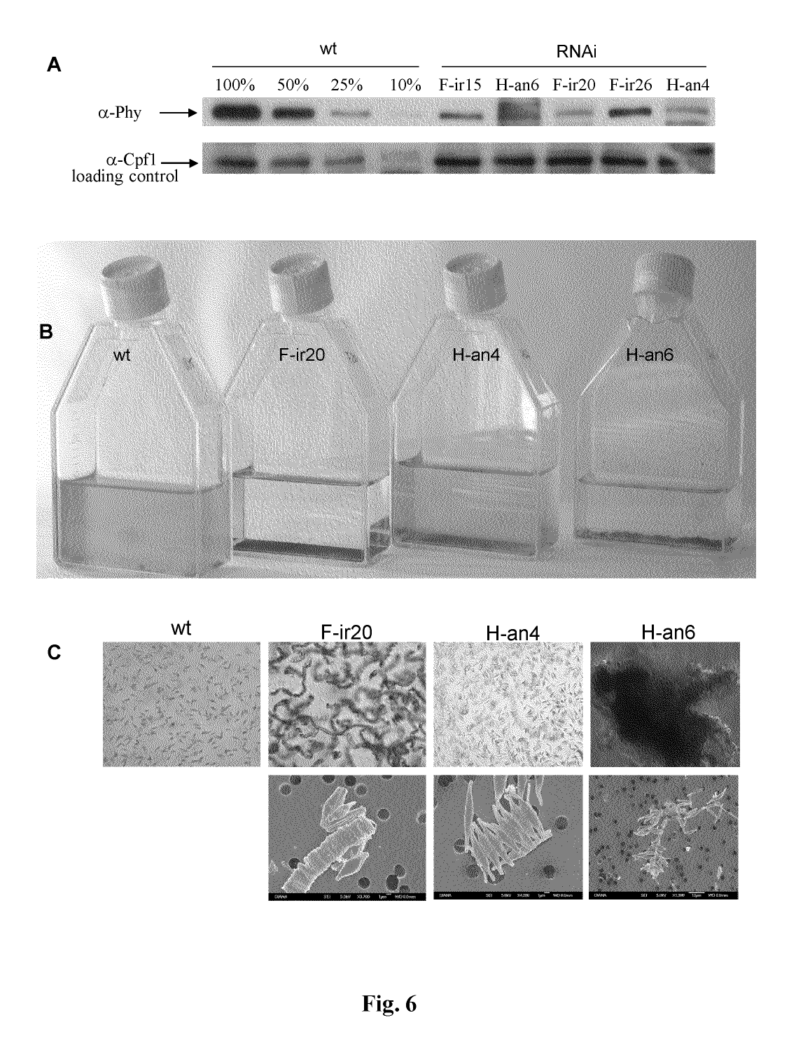

In conclusion, five clones showed a stable and significant reduction in Phy content in comparison to the wild type; in particular, two clones (F-ir15 and F-ir26), transformed with the inverted repeat construct under FcpBp, showed a protein decrease of ca. 50% in comparison to the wild type, two clones, F-ir20 and H-an4 (254 bp) revealed a reduction of ca. 75% and another one (H-an6), containing a 254 bp antisense construct driven by the H4p, showed a decrease of ca. 90% (FIG. 6A). The same results, also in terms of silencing extent, were found for all the five mutants by western blot after one year, demonstrating the stability of silencing.

Comparison of the relative PHY expression levels determined by qRT-PCR in the wild type and in the silenced clones revealed the absence of a significant correlation between the decrease in the protein content of transformed cells and a decline in the PHY mRNA content (data not shown), suggesting a possible inhibition at the translational level (De Riso et al. 2009).

### Characterization of Aggregation Phenotype of Silenced Clones

Interestingly, observations of the most silenced clones (F-ir20 and H-an6, H-an4) highlighted particular phenotypes in comparison to wild type P. tricornutum cells. Already by looking at the mutants grown in liquid medium, it was possible to observe sedimentation of the cells to the bottom of the flask (FIG. 6B). A more accurate analysis by microscopic observation revealed particular morphologies and aggregation phenotypes in the mutants.

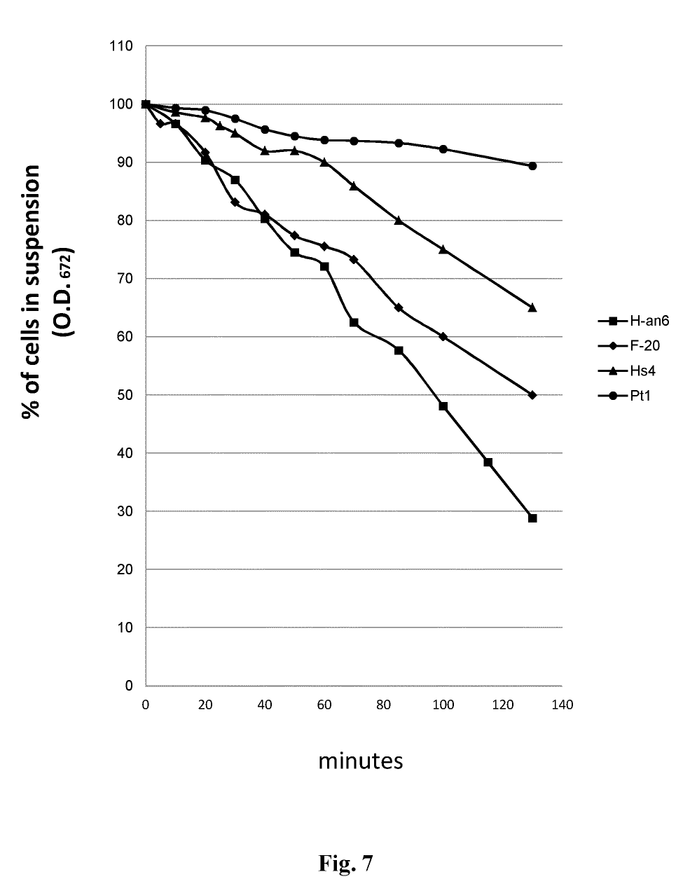

In F-ir20 and H-an6, showing a Phy decrease of respectively 75% and 90%, the cell morphology shifted from the tapering shape typical of wild type cells to a characteristic ovoid shape (FIG. 6C). More interestingly, in all the three more silenced clones (F-ir20, H-an 6 and H-an4) a particular and uncommon aggregation of cells was generated. The F-ir20 and the H-an4 clones showed the generation of chains, with the cells associated along their major axis. In the case of H-an6 the cells were strongly and chaotically aggregated even if in the less dense aggregates also the presence of short cell chains was apparent (FIG. 6). Preliminary analysis suggests that the level of aggregation correlates with cell division, and in fact the chains become longer with increasing division rates. In addition, a higher level of aggregation could be reached by growing the cells under optimal growth conditions. Moreover, investigation by electron microscopy revealed the strong adhesion between the valves of adjacent cells in the chains and the presence of uncharacterized polymeric materials (presumably polysaccharides) around and between cells of H-an6 (FIG. 6 C). In order to better quantify the effect of the PHY silencing on the sedimentation, we followed the optical density variations over the time of the wild-type and the mutants, as the O.D₆₇₂ nm is proportional to the cell in suspension. As shown in FIG. 7, all the mutants showed a sedimentation rate faster than the wild types, and proportional to the silencing level. In particular, after two hour of measurement were still in suspensions: 95% of the wild-type cells, 65% of the H-an4, 50% of F-ir20 and 30% of the H-an6.

### BIBLIOGRAPHIC REFERENCES

Armbrust E V, et al. (2004) Science 306(5693):79-86
Bowler C, et al. (2008) Nature 456(7219):239-44
Carrieri D, et al. (2008) Curr Opin Biotechnol 19(3):235-240 Chen M, Chory J, Fankhauser C (2004) Annu Rev Genet 38: 87-117
Chisti Y (2007) Biotechnol Adv 25(3):294-306.
Chisti, Y (2008) Trends Biotechnol 26:126-131
De Riso V, et al. (2009) Nucleic Acids Res. 1-12 doi:10.1093/nar/gkp448
Dismukes G C, et al. (2008) Curr Opin Biotechnol 19:235-240
Dunstan G A, et al. (1994) Phytochemistry 35:155-161.
Falciatore A and Bowler C (2005) Curr Top Dev Biol 68:317-350
Falciatore A, et al. (1999) Marine Biotech 1:239-251
Falkowski P G, Barber R T and Smetacek V (1998) Science 281:200-206
Guillard R R L (1975) Culture of phytoplankton for feeding marine invertebrates. In Culture of Marine Invertebrates Animals. Edit by Smith W L, Chanley M H, New York, USA.
Lebeau T and Robert J M (2003) Appl Microbiol Biotechnol. 60(6):624-32
Montgomery B L, and Lagarias J C (2002) Trends Plant Sci. 7:357-366.
Montsant A, et al (2007) J of Phycol 43:585-604.
Sambrook J, Fritsch E F, Maniatis T (1989) Molecular cloning: A laboratory manual. Cold Spring Harbor Laboratory Press, Cold Spring Harbor, N.Y.
Siaut M, et al. (2007). Gene 406:23-35.
Vuillet L, et al. (2007) EMBO J 26(14):3322-31
Zaslayskaia L A, et al. (2001) Science 292:2073-2075.

1. A method to induce alga and/or diatom cell bio-flocculation comprising the step of inactivating the expression of at least one photoreceptor in said alga and/or diatom.

2. The method of claim 1 wherein the alga is selected from any of the following groups: the species Chromalveolata and Archaeplastida or the group of Bacillariophyceae.

3. The method of claim 1 wherein the diatom is selected from the species Thalassiosira pseudonana or Phaeodactylum tricornutum.

4. The method of claim 1 wherein the photoreceptor belongs to the group of red light photoreceptors.

5. The method of claim 1 wherein the photoreceptor is a phytochrome.

6. The method of claim 5 wherein the phytochrome is the Phy protein, consisting essentially of SEQ ID. No. 2, or variants thereof.

7. The method of claim 1 wherein the inactivation of the expression of the photoreceptor is achieved by means of genetic engineering.

8. The method of claim 7 wherein the genetic engineering comprises the step of transforming the alga and/or the diatom cell with a nucleic acid construct able to inactivate the expression of the photoreceptor.

9. The method of claim 8 wherein the nucleic acid construct able to inactivate the expression of the photoreceptor comprises an alga and/or diatom cell active promoter operatively linked to a nucleic acid fragment of the photoreceptor cDNA.

10. The method of claim 9 wherein the nucleic acid construct is able to inactivate the expression of the photoreceptor Phy protein, essentially consisting of SEQ ID. No. 2, or variants thereof, comprising a diatom active promoter region and operatively linked to a nucleic acid fragment of the PHY cDNA comprised between nt 727 and nt 980 of SEQ ID No. 1 or between nt 727 and nt 1147 of SEQ ID No. 1 introduced in the antisense orientation with respect to the nucleic acid fragment of the PHY cDNA.

11. The method of claim 10 wherein the promoter region is selected from the promoter region of the Fucoxanthin Chlorophyll a/c-binding Protein B gene (SEQ ID. No. 15) or of the Histone 4 gene (SEQ ID. No. 16).

12. A bio-flocculated alga and/or diatom cell obtainable according to the method of claim 1.

13. The bio-flocculated alga cell according to claim 12 being selected from the species Chromalveolata and Archaeplastida or from the group of Bacillariophyceae.

14. The bio-flocculated diatom cell according to claim 12 being selected from the species Thalassiosira pseudonana or Phaeodactylum tricornutum.

15-17. (canceled)

18. Biodiesel comprising the bio flocculated alga and/or diatom cell according to claim 12.

19. A method of bioremediating contaminated water or water enriched in phosphate and/or nitrogen, comprising: contacting the contaminated water or water enriched in phosphate and/or nitrogen with the bio flocculated alga and/or diatom cell according to claim 12.

20. A livestock or poultry feed comprising the product of claim 12.


SEQUENCE LISTING (17 sequences)

SEQ ID NO: 1 - DNA, 3033 bp, Phaeodactylum tricornutum
  CDS (1)..(3033)
atgagcggggcaaattatagagaagccgacttccctggtgtccgtgccgcgggtcggcacaacaattccattacaaccaaagagctgacggaatgtgatcgtgagcctgtgcacttgatcgcaaacgtacaagggggtaccggccatttgttgttcattcactacccgtctggaaaaatcttggctcatgatcgcgacatcgaacacattccttggatccggtgtcacgaaaacagaacagttaccgctggccgcactggggcgagaactacatcttctttccatggagaacagcagagtggtgagagtcctcacgaagctattggcatatctggaggctttttactgaactgggttccgcacgatttctacgagaagattctcgatttggtcctcggtattatccattccgatacgcacagaaatttttatttttattcatatgatggttcagcgtacgctatttctatttcagcgacggaaatggactactccgtgattggcatcgaaattgaaacagttggtttggatgatactgcctcccatttttcatcctcattgttgcatttgggacgtattgtggaattctaccagcacgaagcaattgccaagacagcctgtgacactgtttttcacctattgggaaagtatgacaggggcatggtgtaccgattccacgatgatctgtccggcgaggtcgtgcacgagattaaagcaaatcatgtggaatccagctatcttggcatgcgatttccttcctctgatattcctttgccatcgcgacagctttatataaaaaatggtgtgcggtacatttacgacgttgataccgaggatctaccgattttatccctggacaatgaaaagatggatctcagtcaaattcgcatgcgtgctgtagccaaaccgcatattgtgtacttaagaaatatgggggtggtgtcgtcgttgagcttggcgattgttgtcgacaatgatctgtgggggttgctggcttttcatgggtacggcgcgaggtacaagccttcgctccatcagcgaattgcttgtgaaaccataagtgcgatggtctcagttcgtattgaatctctcatgaaaaaggcgcagagtgcccgaattattaagttgggccagtgcactatgagcttaaagcatgaccagagcctgattcacaatctctatgaatggggtgaaggcatactcgaaattgttgatggagatgttttggttgcacatatacaagatcctagagatggcgaaggcgacagaattgtgctgggtgatcctttgttggtaccgaaggattctttttggactaagatgagttcctatcagaatcgcgaactctgtgtcatttcaacacgcaaagctctcacagatatcaaattgacacaagaagagtgcccagcaagtggaattgtatttttccaagagggtcgtactcagatcatgattggacgagcaatgcgatccaaagatgtcgtatgggcaggtaatcctgacgaaccaaaactaaggattggaggaattttgaatccgcgcaactcctttactcaattcattgaaaaagcgcgaaaggaatcacgagcctggactgtgcaagatattagtgtgatttctgtgcttcgtgaccgtatatgtgagcattcgtacgcatacatgatgggattactgagaggtgatattcaagatgcaaaccggaaatatttggcggcaattgacagagcgcgggacaattacgaattctttgcgcatatgagccacgaactacggactcctttccatggcgttatgggatgcttaagtattctgcatgagtcaattgaagatatgccagcagcggaagtcagagatgttgtcgatacagcaatagcttccggaaaccacatgatcaatcttctcaacgatattctggacatctcgaagaacaaacacttgtctcatatatcggcgcaggataaggttatttaccagactttagcctttgagacaattgactgtatgaagtcactggccacgtcgcgaaagatcgagatgagatcgtcaatcgagccgaaaggtttggaaaaagtggtgattgtgacggatcgtacaaaaattattcaaatcgtttccaacgttgtgaacaatgccatcaagtttacgggtgaagggactgtcgatgttgtatttaggctcgttgattcgctgcaagaggcaactatgatgtgggagcgaggcgcggaagttcatgctggatcagtgttttcgatgaaggagagtgaaatgcacacatcggctgaagaagtaagacggagcaccatgacgtttaatgagacgcatgatcaaaagtggatgacaatgagtgtctcagacaccggatgcggtatggagccgtctgaactagtagaaatgttctcaccatatacccaatcgagtcatggatccaatcgcatttttcagggaacagggcttgggcttttcatttgcgtttcattatgttaccaactcaatggttttatttcttgtgcgagcacccccgataaaggaacactttttcatatgggaatcccagtcggattgttagctgaagacacagttgagggaaatcagacactaacagatgatacgaaggaaacagagagcgtgatccaaatgtcgggtccgattttgatcgtagatgacaatgttgtgaacgtgaaaattctaaaccgggcgctacttttggatattagaagagctggtcttgcaatagaggtcctcacagcagggggtggggctgaaggtgtccaggtctttcgagacaagcgccccagtctatgcattatcgactatcacatgcccgatgtcgatggcattgaagcgacctgcaccatacggaaatacgagcaagaaaacaaaattgatcctacctacattttgatgtacactgctgatgccacagagcaagctagagcattgatcttgagctccggcgttgacgatatcatgtccaagcctccgccgaagggattcattgccgggttggtgcagaggctgcgggttccggaatag

SEQ ID NO: 2 - protein, 1010 aa, Phaeodactylum tricornutum
MSGANYREADFPGVRAAGRHNNSITTKELTECDREPVHLIANVQGGTGHLLFIHYPSGKILAHDRDIEHIPWIRCHENRTVTAGRTGARTTSSFHGEQQSGESPHEAIGISGGFLLNWVPHDFYEKILDLVLGIIHSDTHRNFYFYSYDGSAYAISISATEMDYSVIGIEIETVGLDDTASHFSSSLLHLGRIVEFYQHEAIAKTACDTVFHLLGKYDRGMVYRFHDDLSGEVVHEIKANHVESSYLGMRFPSSDIPLPSRQLYIKNGVRYIYDVDTEDLPILSLDNEKMDLSQIRMRAVAKPHIVYLRNMGVVSSLSLAIVVDNDLWGLLAFHGYGARYKPSLHQRIACETISAMVSVRIESLMKKAQSARIIKLGQCTMSLKHDQSLIHNLYEWGEGILEIVDGDVLVAHIQDPRDGEGDRIVLGDPLLVPKDSFWTKMSSYQNRELCVISTRKALTDIKLTQEECPASGIVFFQEGRTQIMIGRAMRSKDVVWAGNPDEPKLRIGGILNPRNSFTQFIEKARKESRAWTVQDISVISVLRDRICEHSYAYMMGLLRGDIQDANRKYLAAIDRARDNYEFFAHMSHELRTPFHGVMGCLSILHESIEDMPAAEVRDVVDTAIASGNHMINLLNDILDISKNKHLSHISAQDKVIYQTLAFETIDCMKSLATSRKIEMRSSIEPKGLEKVVIVTDRTKIIQIVSNVVNNAIKFTGEGTVDVVFRLVDSLQEATMMWERGAEVHAGSVFSMKESEMHTSAEEVRRSTMTFNETHDQKWMTMSVSDTGCGMEPSELVEMFSPYTQSSHGSNRIFQGTGLGLFICVSLCYQLNGFISCASTPDKGTLFHMGIPVGLLAEDTVEGNQTLTDDTKETESVIQMSGPILIVDDNVVNVKILNRALLLDIRRAGLAIEVLTAGGGAEGVQVFRDKRPSLCIIDYHMPDVDGIEATCTIRKYEQENKIDPTYILMYTADATEQARALILSSGVDDIMSKPPPKGFIAGLVQRLRVPE

SEQ ID NO: 3 - DNA, 25 bp, Artificial Sequence
  synthetic primer
actgaatccagctatcttggcatgc

SEQ ID NO: 4 - DNA, 26 bp, Artificial Sequence
  synthetic primer
acttctagatcattgtcgacaacaat

SEQ ID NO: 5 - DNA, 26 bp, Artificial Sequence
  synthetic primer
gattctagagctcatagtgcactggc

SEQ ID NO: 6 - DNA, 26 bp, Artificial Sequence
  synthetic primer
agtccgcggaatctcgcctattcatg

SEQ ID NO: 7 - DNA, 20 bp, Artificial Sequence
  synthetic primer
gcgtacgctgaaccatcata

SEQ ID NO: 8 - DNA, 20 bp, Artificial Sequence
  synthetic primer
gttccgcacgatttctacga

SEQ ID NO: 9 - DNA, 20 bp, Artificial Sequence
  synthetic primer
gaattgtgctgggtgatcct

SEQ ID NO: 10 - DNA, 20 bp, Artificial Sequence
  synthetic primer
gtgagagctttgcgtgttga

SEQ ID NO: 11 - DNA, 20 bp, Artificial Sequence
  synthetic primer
gacatcgggcatgtgatagt

SEQ ID NO: 12 - DNA, 21 bp, Artificial Sequence
  synthetic primer
gcaatagaggtcctcacagca

SEQ ID NO: 13 - DNA, 21 bp, Artificial Sequence
  synthetic primer
gtgcaagagaccggacatacc

SEQ ID NO: 14 - DNA, 21 bp, Artificial Sequence
  synthetic primer
cgaagtcaaccaggaaaccaa

SEQ ID NO: 15 - DNA, 437 bp, Phaeodactylum tricornutum
aatctcgcctattcatggtgtataaaagttcaacatccaaagctagaacttttggaaagagaaagaatatccgaatagggcacggcgtgccgtattgttggagtggactagcagaaagtgaggaaggcacaggatgagttttctcgagacacatagcttcagcgtcgtgtaggctaggcagaggtgagttttctcgagacataccttcagcgtcgtcttcactgtcacagtcaactgacagtaatcgttgatccggagagattcaaaattcaatctgtttggacctggataagacacaagagcgacatcctgacatgaacgccgtaaacagcaaatcctggttgaacacgtatccttttgggggcctccgctacgacgctcgctccagctggggcttccttactatacacagcgcgcatatttcacggttgccagaa

SEQ ID NO: 16 - DNA, 650 bp, Phaeodactylum tricornutum
gcaatctcacgcaccaggcgctggaagggcaacttgcggatgagaaggtccgtggacttctggtaacgacggatctcacgcagagcgacggttccagggcgataacggtggggcttcttgactcctccggtagccggagcggacttgcgggcagccttggtggcaagctgcttgcgcggcgctttgcctccggtggatttacgggcggtttgcttggttcgggccattttgacggttttttttacaagagaagagttcttgaaatttgtgaggttaaagtgtgtggcttccgccgtagtcaaggagcgtgcggttgccgatcgcaccggtacgttctgtagaaatgaacacagtgtgttgaattgaaagtatggcgcaggtatggtgtgtgataagtagcagccgcgccgagacaaacaaactttggtttctacgacaatctctgtagacaagtactagaaacccgtttgaacgagcataaatctgcaccggcaggccaccagacatcgtttcaacgtaatattctacgtaaccattttatcccaggaaacctacggcctgtgaaccaccgagacggagcactcacaattcgctctcggcaacaaccgacaatcgtcttactcacagtcaataccgaaaacaaacaacag

SEQ ID NO: 17 - protein, 953 aa, Thalassiosira pseudonana
MIGKPLGLIFGAEHVSQVKDVIENVIMTSGGGGGSASSSGERHSLPSFIQPPSKRRVSDDNFLLRSESTLSLSGRISCSVLPSSQNFLLELEKTPLLEKYTQFEDRDVMSFMEEIAKELRACWSIEEMASLVCAKVMQETPYDRGMVYKFDHEDCGEVVYEAFRSDASEACRKDSFLGLRFPASDIPRQARELFMRNTLRVVYDVDGNDFELYPPMVDIKRASGEKELGYTDLSMCRLRGSSFVHLKYLKNMGVTSTMVIAIIVNGRLWGLYSFHGYREPLVPSARTRFLCEMASITTSIIMESLTRKESNERLMSLDSLMNSLQTTSLSNFVETNLSDIMGALQVNLISFRVRDPPATPEIKTFLDETDGKMESPDEITNEVFDSLVETYGSVCRDYGVVYIDDQKSNTLLVNRGIHTLVFFRTSGVDVILSRNRTIERVTWGGDPDKQLEPDGTLTPRNSFAAYVEDHFKKGKPWDATDRQLISRFSDQLEKHRTKELNLEHSKTIKTLEKEKAEATETARVNFDFFAHMAHELRTPFHGMVGSLEAMREDPVLRNNEMLKTAELCGKSMIKILDDILLVAKGSYDLQIEEHLFDLKSFVKITANDMSSFALMEGAPIQLGKELIFCKDLVGDSQRIRQVMNNLLSNAIKFSEDSISLDVVQKNSFSEVLAVWKTYSNVYPNCEPTLLELSTFVAEASEASVDTIWVIFSIVDKGIGITGPDLKKLGTAFTQLSSGRQKKYQGTGLGLNICNMLVGALGGKLVMFSAKGLGSCFTFAVPVKKSDVPVAEPEVEESQNEKAKKMEALQEEFDAFGFREKGVRIFVVDDSAINRKLCGRKIRRWMPSVTIKECSSGSSALAEYEKSPKDVMGIFMDYHMQDMDGDECTRRIRECESMREDLPNVYIVGYTADVLEDSTGRLMESGMNSVMPKPEPPRGECDSKLSIRKTSRPL